(19) United States
(12) Patent Application Publication
(10) Pub. No.: US 2005/0027295 A1
(43) Pub. Date: Feb. 3, 2005

(54) Interlocking nail

(76) Inventor: Lin Min Yang, Taichung (TW)

Correspondence Address: Liberty Patent and Trademark Office, P.O. Box 590, Taichung City, Taichung, 400, TW

(21) Appl. No.: 10/631,719

(22) Filed: Aug. 1, 2003

(51) Int. Cl. A61B 17/56

(52) U.S. Cl. 606/62

(57) ABSTRACT

An improved interlocking nail has an accommodating space at one end of main unit, and an inclined side on one side of the accommodating space, with a plug installed inside the accommodating space to match the inclined side. When the main unit of the improved interlocking nail is implanted into the bone, the main unit will not extend beyond the bone, thereby reducing pain to the patient. The plug which designed in specified colors indicates the location of the interlocking nail, thereby facilitates an interlocking nail removing process.

### BACKGROUND OF THE INVENTION

(a) Field of the invention

The invention relates to an improved interlocking nail that is implanted into a fractured bone for supporting and fixation purposes.

(b) Description of the Prior Art

To enable effective healing of a fractured bone, it is a common practice to implant an interlocking nail into the bone. As shown in FIG. 1 that illustrates how the interlocking nail is implanted into the bone. The main unit 11 is implanted axially into the bone 12. A locking screw 13 is inserted between the main unit 11 and the bone, tightening the main unit 11 to the bone 12 in secured relative positions.

To facilitate removing the interlocking nail, generally the main unit 11 is not fully implanted into the bone 12. Instead, a protrusion 14 is reserved outside the bone 12. The protrusion 14 of the interlocking nail for indicating the location where removing the interlocking nail later. However, the protrusion 14 of interlocking nail will keep on irritating the patellar tendon, resulting in pain. Alternatively, if the main unit 11 of the interlocking nail is completely implanted into the bone 12, the overall surface of the bone 12 will be overlapped by new-grown bone tissues, in order to locate the interlocking nail for then removing it, it is necessary to sacrifice some new-grown bone, this results secondary injury. Therefore, it is desirable to make improvement on the conventional interlocking nails.

### SUMMARY OF THE INVENTION

The present invention relates to an improved interlocking nail comprises a main unit and a plug, the main unit with an inclined side at one end, so the main unit can be implanted into the bone completely. Meanwhile, the main unit can be easily located during removing process.

To achieve the above purposes, one end of the main unit has an accommodating space. One side of the accommodating space is an opening, one side of the opening being an inclined side. Thereby the plug is installed inside the accommodating space and aligned with the inclined side, thus the main unit can be fully implanted into the bone without any extension outside the bone. The plug on the main unit then serves as an indication to locate the interlocking nail.

### BRIEF DESCRIPTION OF DRAWINGS

FIG. 1 is a prior art in use.

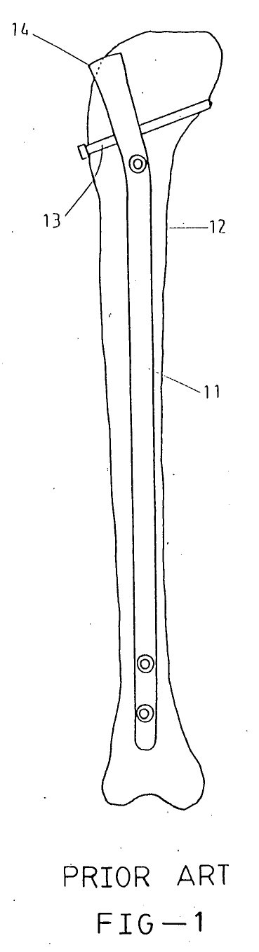

FIG. 2 is an exploded view of the present invention.

FIG. 3 is a first view of the invention in use.

FIG. 4 is a second view of the invention in use.

FIG. 5 is a partial view of the invention.

### BRIEF DESCRIPTION OF NUMERALS

| | |
|---|---|
| 11 main unit | 12 bone |
| 13 locking screw | 14 protrusion |
| 21 main unit | 22 first end |
| 23 second end | 24 accommodating space |
| 25 opening | 26 inclined side |
| 27 flange | 31 plug |
| 32 face | 33 insert |

### DETAILED DESCRIPTION OF PREFERRED EMBODIMENTS

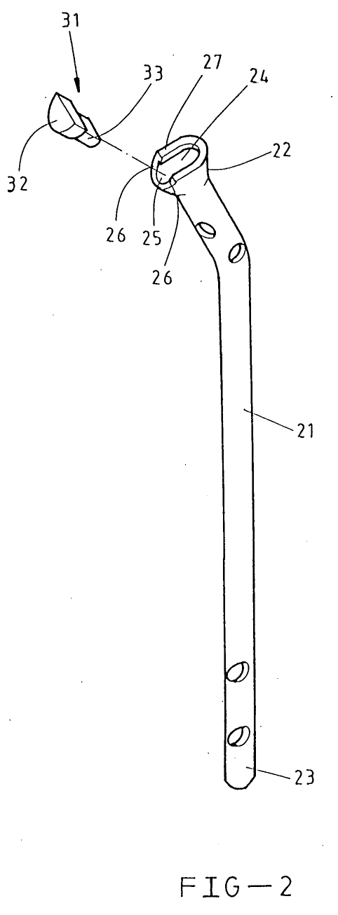

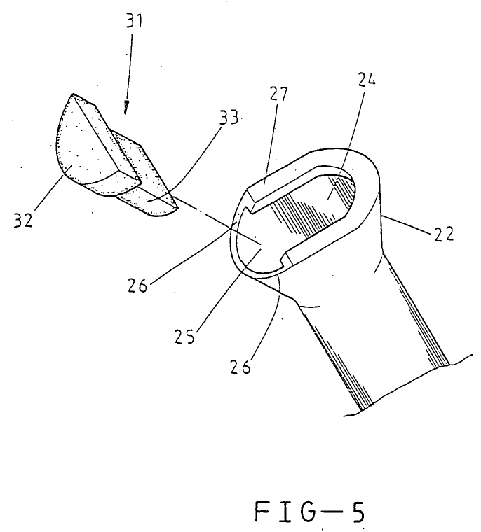

FIGS. 2 and 5 illustrate a main unit 21 of an interlocking nail, having a first end 22 and a second end 23 far away from the first end 22. The first end 22 has an accommodating space 24. At one side of the accommodating space 24 is an opening 25. The opening 25 has an inclined side 26. The top of accommodating space 24 shaped as a flange 27.

A plug 31 is made to have a face 32, and an insert 33 that is attached to the face 32. The size of face 32 is larger than the insert 33. The entire unit and the face 32 of the plug 31 are in bright colors.

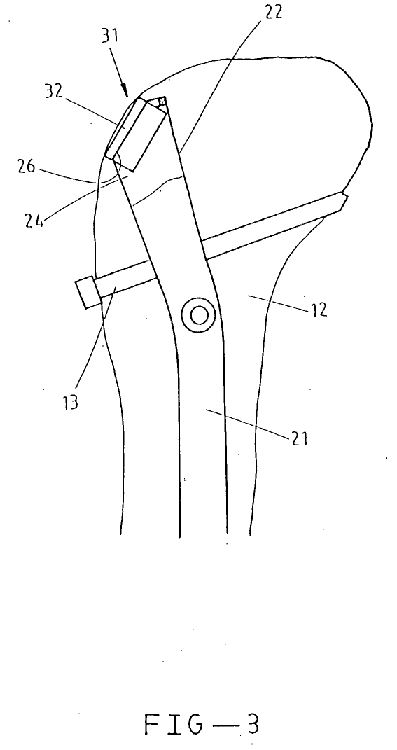

FIG. 3 illustrates an application of present invention, wherein the main unit 21 is implanted into the bone 12 and secured by a positioning nail 13. The plug 31 is installed inside the accommodating space 24 at the first end 22 of the main unit 21. Because the particular designed inclined side 26 of the accommodating space 24, and the face 32 of the plug 31 also rest on the inclined side 26, the main unit 21 has no protrusion extending beyond the end of the bone 12 when the interlocking nail implanted. Thereby, the patient suffers no pain that may be caused by interlocking nail.

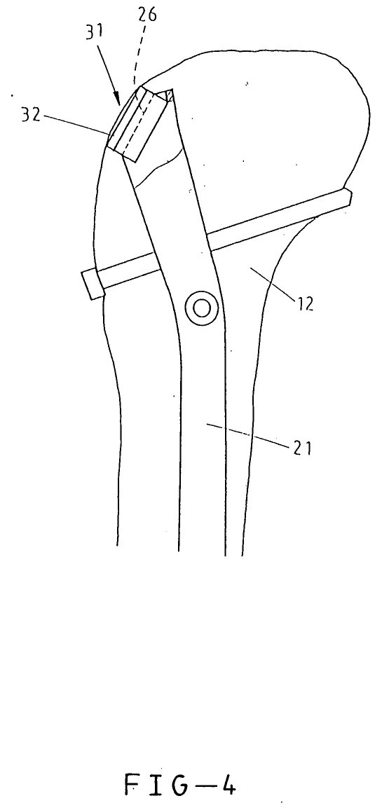

As shown in FIG. 4, when the main unit 21 is implanted to an excessive depth into the bone 12, the plunging depth of the plug 31 to the accommodating space 24 can be adjusted, in order to make the face 32 of the plug 31 close to the surface of the bone 12. Therefore, when in removing the interlocking nail, the position of the main unit 21 can be located precisely by finding the plug 31 without to cut the muscles for finding it, thereby reducing subsequent pain of the patient.

When in the process of removing the main unit 21, the present invention not only provides the function of the plug 31 for indicating the location of the main unit 21, since there is an opening 25 on one side of the accommodating space 24 and flange 27 at the top of the accommodating space 24, a removing tool of main unit 21 can be inserted conveniently and rapidly along the opening 25 into the accommodating space 24 and aligned with the flange 27. Then, the removing tool is engaged to the flange 27 to pull out the main unit 21 smoothly.

Thus, present invention has a simplified design to ensure no protrusion of the first end 22 of the main unit 21 beyond the end of the bone when the main unit 21 is implanted inside the bone, thereby the patient feels no pain, nor in causing the removing problem when the interlocking nail implanted in a excessive depth. Therefore, the inventive step of the present invention has effectively overcome the problems of conventional art whether the main unit is implanted too deep or too shallow into the bone.

The foregoing covering the preferred embodiments and drawings of the present invention are for the purpose of description, and should not be used to limit the scope of the claim of the present invention. It is to be understood that all equivalent modifications made without departing from the spirit of the following claim shall be included in the subject claim.

1. An improved interlocking nail, comprising:
a main unit, having a first end, and a second end that is far away from the first end, said first end having an accommodating space, on one side of said accommodating space being an opening, said opening having an inclined side;
a plug, having a face, and a insert in connection with the face, the plug installed by inserting inside the accommodating space of the main unit.

2. The improved interlocking nail in claim 1, wherein the accommodating space of the main unit has a flange on a top rim.
